(19) United States
(12) Patent Application Publication
(10) Pub. No.: US 2023/0401736 A1
(43) Pub. Date: Dec. 14, 2023

(54) METHOD FOR PROVIDING CENTER-OF-GRAVITY INFORMATION BY USING IMAGE, AND APPARATUS THEREFOR

(71) Applicant: KAKAO VX CO., LTD, Seongnam-si, Gyeonggi-do (KR)

(72) Inventors: Kwang-Jun LEE, Seongnam-si, Gyeonggi-do (KR); Dong-Jin SHIN, Seongnam-si, Gyeonggi-do (KR)

(21) Appl. No.: 18/033,445

(22) PCT Filed: Sep. 23, 2021
(86) PCT No.: PCT/KR2021/012942

§ 371 Date: Apr. 24, 2023

(30) Foreign Application Priority Data
Oct. 27, 2020 (KR) 10-2020-0140532

(51) Int. Cl. G06T 7/66; G01M 1/12

(52) U.S. Cl. CPC G06T 7/66; G01M 1/122

(57) ABSTRACT

A method for providing center-of-gravity information by using an image, and an apparatus therefor are disclosed. A method for providing center-of-gravity information, according to one embodiment of the present invention, comprises: generating multi-angle two-dimensional poses corresponding to a user's pose on the basis of images of the user captured at multiple angles; generating a three-dimensional pose by combining the multi-angle two-dimensional poses; generating, on the basis of the three-dimensional pose, a heatmap visually showing the pressure distribution of soles; extracting the center point of gravity on the basis of the heatmap; and providing center-of-gravity information corresponding to the user's pose by using a weight balance calculated on the basis of the center point of gravity.

### TECHNICAL FIELD

The present disclosure relates generally to a method for providing center-of-gravity information using an image and an apparatus for the method, and more particularly to technology for providing information related to the front/rear and left/right center-of-gravity of a person who is included in captured images by analyzing images captured by a person from various angles.

This application claims the benefit of Korean Patent Application No. 10-2020-0140532, filed Oct. 27, 2020, which is hereby incorporated by reference in its entirety into this application.

### BACKGROUND ART

In many sports, the location or shift of the center of gravity acts as an important factor in improving ability. For example, in golf, the degree to which the center of gravity of a body is shifted and the timing at which the center of gravity is shifted may act as important factors in assuming a correct swing posture.

However, in order to measure the center of gravity of a person and the shift of the center of gravity, separate equipment, such as a floor mat including a pressure measurement sensor or exclusive measurement shoes personally worn by a user, is required. This special equipment has various restrictions such as the occurrence of high initial cost to manufacture and purchase the specific equipment, the occurrence of additional cost to charge a battery or pay damage repair cost, and limitation in a place depending on the scheme for measuring the center of gravy.

Furthermore, such exclusive measurement equipment is limited in that it cannot be freely used in a mobile platform, and platforms provided by equipment manufacturers need to be used.

### DISCLOSURE

### Technical Problem

An object of the present disclosure is to provide center-of-gravity information related to the posture and balance of a user using only images captured by a camera in a usual place in which separate measurement equipment is not provided without restrictions in a place or a device.

Another object of the present disclosure is to provide a center-of-gravity information service in the form of an application freely executable in an environment such as in a user's individual PC or a smartphone.

A further object of the present disclosure is to provide a service that assists users who enjoy sports influenced by exercise posture or the shift of the center of gravity in correcting their postures or in more efficiently moving by providing center-of-gravity information to correspond to the motion of each user included in images.

Yet another object of the present disclosure is to provide a service that can utilize exercise indices such as posture correction, the accuracy of posture, and the timing of weight shifting, or to provide service technology that can record the degree of correctness in exercise posture as an index and utilize the index in such a way as to monitor the index and that can provide a feedback in various sports fields such as not only ball games such as golf or baseball but also gymnastics or freehand exercise.

### Technical Solution

In accordance with an aspect of the present invention to accomplish the above objects, there is provided a method for providing center-of-gravity information, including creating multi-angle two-dimensional (2D) poses corresponding to posture of a user based on images obtained by capturing the user from various angles; creating a three-dimensional (3D) pose by combining the multi-angle 2D poses with each other; generating a heatmap for visually showing a plantar pressure distribution based on the 3D pose, and extracting a midpoint on a weight balance based on the heatmap; and providing center-of-gravity information corresponding to the posture of the user using a weight balance calculated based on the midpoint on the weight balance.

Here, the weight balance may include a front/rear weight balance and a left/right weight balance.

Here, the method may further include outputting the center-of-gravity information to correspond to the images based on a user interface.

Here, the outputting may include outputting at least one of the heatmap, the weight balance, the midpoint on the weight balance, and a shift line of the midpoint on the weight balance.

Here, generating the multi-angle 2D poses may include converting each image into array data composed of 8-bit integers; and representing multiple joints corresponding to the posture of the user in a 2D coordinate system by inputting the array data into a deep learning-based joint estimation model.

Here, the multi-angle 2D poses may correspond to data represented by connecting the multiple joints to each other in the 2D coordinate system.

Here, extracting the midpoint on the weight balance may include generating the heatmap by inputting the 3D pose to a deep learning-based center-of-gravity estimation model.

Here, extracting the midpoint on the weight balance may include extracting the midpoint on the weight balance by normalizing output values from the heatmap.

Here, providing the center-of-gravity information may include generating a correction value for the midpoint on the weight balance; calculating a left/right weight balance of a left foot and a right foot of the user based on the correction value for the midpoint on the weight balance; and calculating front/fear weight balances corresponding to the left foot and the right foot by normalizing values corresponding to the left foot and the right foot, respectively, in the heatmap to extract a midpoint on a weight balance of the left foot and a midpoint on a weight balance of the right foot and by correcting respective extracted midpoints.

Here, the images captured from various angles may include a front image obtained by capturing the user from front and a side image obtained by capturing the user from side.

Here, converting into the array data may include extracting valid frames by deleting unnecessary frames from all frames corresponding to the images, and converting the valid frames into the array data.

Here, the heatmap may be displayed to be visually highlighted as pressure applied to soles is higher.

In accordance with another aspect of the present invention to accomplish the above objects, there is provided an apparatus for center-of-gravity information, including a processor configured to create multi-angle two-dimensional (2D) poses corresponding to posture of a user based on images obtained by capturing the user from various angles, create a three-dimensional (3D) pose by combining the multi-angle 2D poses with each other, generate a heatmap for visually showing a plantar pressure distribution based on the 3D pose, extract a midpoint on a weight balance based on the heatmap, and provide center-of-gravity information corresponding to the posture of the user using a weight balance calculated based on the midpoint on the weight balance; and a memory configured to store at least one of the images, the heatmap, and the center-of-gravity information.

Here, the weight balance may include a front/rear weight balance and a left/right weight balance.

Here, the processor may be configured to output the center-of-gravity information to correspond to the images based on a user interface.

Here, the processor may be configured to output at least one of the heatmap, the weight balance, the midpoint on the weight balance, and a shift line of the midpoint on the weight balance.

Here, the processor may be configured to convert each image into array data composed of 8-bit integers and represent multiple joints corresponding to the posture of the user in a 2D coordinate system by inputting the array data into a deep learning-based joint estimation model.

Here, the multi-angle 2D poses may correspond to data represented by connecting the multiple joints to each other in the 2D coordinate system.

Here, the processor may be configured to generate the heatmap by inputting the 3D pose to a deep learning-based center-of-gravity estimation model.

Here, the processor may be configured to extract the midpoint on the weight balance by normalizing output values from the heatmap.

Here, the processor may be configured to generate a correction value for the midpoint on the weight balance, calculate a left/right weight balance of a left foot and a right foot of the user based on the correction value for the midpoint on the weight balance, and calculate front/fear weight balances corresponding to the left foot and the right foot by normalizing values corresponding to the left foot and the right foot, respectively, in the heatmap to extract a midpoint on a weight balance of the left foot and a midpoint on a weight balance of the right foot and by correcting respective extracted midpoints.

Here, the images captured from various angles may include a front image obtained by capturing the user from front and a side image obtained by capturing the user from side.

The processor may be configured to extract valid frames by deleting unnecessary frames from all frames corresponding to the images, and convert the valid frames into the array data.

The heatmap may be displayed to be visually highlighted as pressure applied to soles is higher.

### Advantageous Effects

According to the present disclosure, center-of-gravity information related to the posture and balance of a user may be provided using only images captured by a camera in a usual place in which separate measurement equipment is not provided without restrictions in a place or a device.

Further, the present disclosure may provide a center-of-gravity information service in the form of an application freely executable in an environment such as in a user's individual PC or a smartphone.

Furthermore, the present disclosure may provide a service that assists users who enjoy sports influenced by exercise posture or the shift of the center of gravity in correcting their postures or in more efficiently moving by providing center-of-gravity information to correspond to the motion of each user included in images.

Furthermore, the present disclosure may provide a service that can utilize exercise indices such as posture correction, the accuracy of posture, and the timing of weight shifting, or may provide service technology that can record the degree of correctness in exercise posture as an index and utilize the index in such a way as to monitor the index and that can provide a feedback in various sports fields such as not only ball games such as golf or baseball but also gymnastics or freehand exercise.

### DESCRIPTION OF DRAWINGS

FIG. 1 is an operation flowchart illustrating a method for providing center-of-gravity information using an image according to an embodiment of the present disclosure;

FIGS. 2 to 4 are diagrams illustrating an example of a process of generating center-of-gravity information based on the front image of a user according to an embodiment of the present disclosure;

FIGS. 5 to 9 are diagrams illustrating an example of a process of generating center-of-gravity information based on the front and side images of a user according to an embodiment of the present disclosure;

FIG. 10 is a diagram illustrating an example in which valid frames are extracted according to the present disclosure;

FIGS. 11 and 12 are diagrams illustrating an example of a user interface for providing center-of-gravity information according to the present disclosure;

FIGS. 13 and 14 are diagrams illustrating in detail a process of providing center-of-gravity information according to an embodiment of the present disclosure; and

FIG. 15 is a block diagram illustrating an apparatus for providing center-of-gravity information according to an embodiment of the present disclosure.

### BEST MODE

The present disclosure will be described in detail below with reference to the accompanying drawings. Repeated descriptions and descriptions of known functions and configurations which have been deemed to make the gist of the present disclosure unnecessarily obscure will be omitted below. The embodiments of the present disclosure are intended to fully describe the present disclosure to a person having ordinary knowledge in the art to which the present disclosure pertains. Accordingly, the shapes, sizes, etc. of components in the drawings may be exaggerated to make the description clearer.

Hereinafter, preferred embodiments of the present disclosure will be described in detail with reference to the attached drawings.

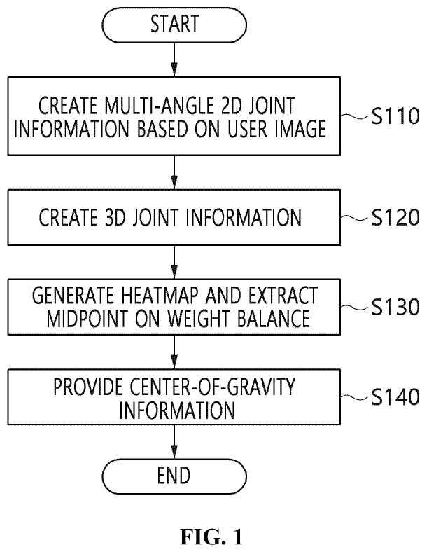

FIG. 1 is an operation flowchart illustrating a method for providing center-of-gravity information using an image according to an embodiment of the present disclosure.

Referring to FIG. 1, the method for providing center-of-gravity information using an image according to the embodiment of the present disclosure creates a multi-angle two-dimensional (2D) pose corresponding to a user posture based on images (video) obtained by capturing a user from various angles at step S110.

That is, the present disclosure may create a 2D pose used to generate center-of-gravity information by utilizing only images captured through a camera even in a usual place in which separate center-of-gravity measurement equipment is not provided.

For example, when the user desires to know whether his or her posture or balance is correct while doing workouts at home, the user may capture images of his or her home workout postures using a camera provided in the smartphone of the user, and may input the captured home workout images through a user interface provided to the smartphone according to an embodiment of the present disclosure.

Here, the images captured from various angles may include a front image obtained by capturing the user from the front and a side image obtained by capturing the user from the side.

Therefore, a multi-angle 2D pose may include a 2D pose created to correspond to the front posture of the user extracted from the front image and a 2D pose created to correspond to the side posture of the user extracted from the side image.

Here, in the present disclosure, the left/right weight balance of the user may be estimated using the 2D pose created to correspond to the front posture of the user.

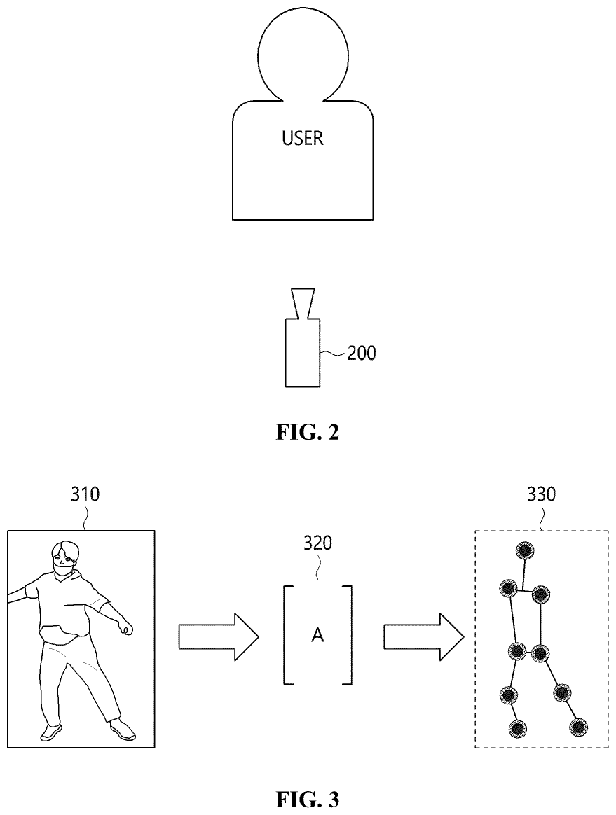

For example, as illustrated in FIG. 2, when the user is captured using one camera 200 located in front of the user, an image 310 including only the front posture of the user may be acquired, as illustrated in FIG. 3. Here, through a process of converting the image 310 into array data 320, which will be described later, a 2D pose 330 corresponding to the front posture of the user may be created.

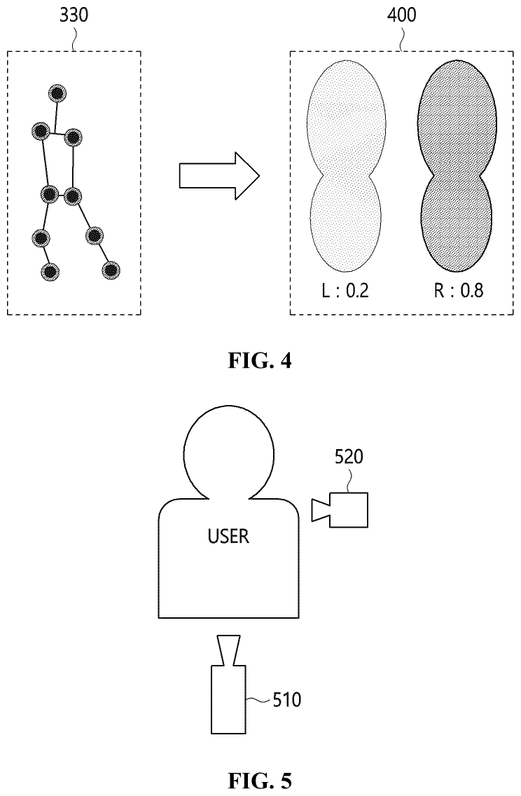

When the 2D pose 330 created in this way is used, the left/right weight balance 400 of the front posture of the user may be estimated, as illustrated in FIG. 4. A process of estimating the left/right weight balance using the 2D pose corresponding to the front posture of the user will be described in detail later.

Here, the left/right weight balance 400 illustrated in FIG. 4 may be a value estimated based on the feet of the user captured in the image 310, illustrated in FIG. 3, and the balance of a right foot and the balance of a left foot may be estimated to be values in inverse proportion to each other.

For example, when the body of the user is analyzed as tilting to the right based on the 2D pose, the balance value of the left foot may be decreased instead of the balance value of the right foot being increased. On the other hand, when the body of the user is analyzed as tilting to the left based on the 2D pose, the balance value of the right foot may be decreased instead of the balance value of the left foot being increased.

Further, in the present disclosure, the front/rear weight balance of the user may also be estimated using the 2D pose created to correspond to the side posture of the user.

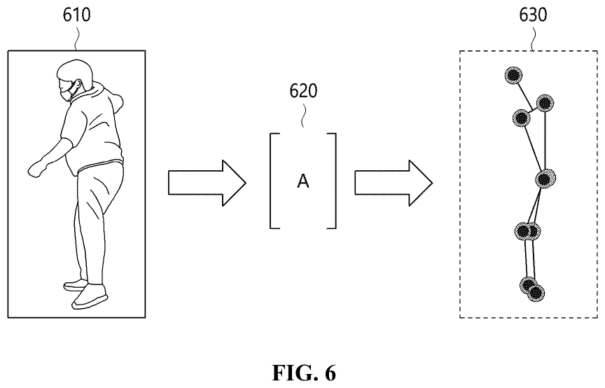

For example, as illustrated in FIG. 5, when the user is simultaneously captured using a camera 510 located in front of the user and a camera 520 located on the side of the user, an image 610 corresponding to the side posture of the user at the same time point, as shown in FIG. 6, may be acquired together with the image 310 corresponding to the front posture of the user, shown in FIG. 3. In this case, when the image 610 corresponding to the side posture of the user undergoes the process of conversion into array data 620, which will be described later, a 2D pose 630 corresponding to the side posture of the user may be created.

That is, a multi-angle 2D pose in the present disclosure may be a concept including both the 2D pose 330 corresponding to the front posture, illustrated in FIG. 3, and the 2D pose 630 corresponding to the side posture, illustrated in FIG. 6.

Here, in order to create the multi-angle 2D pose, the positions of respective joints of the user need to be estimated by inputting images captured from various angles to a deep learning-based joint estimation model, wherein, for this operation, the images captured from various angles may be first converted into array data to be input to the deep learning-based joint estimation model.

Here, the joint estimation model may correspond to a deep learning-based model.

For example, the joint estimation model according to an embodiment of the present disclosure may correspond to a deep learning model designed by utilizing a TensorFlow framework.

Here, the array data may be data composed of 8-bit integers.

Here, the images converted into the array data may be input to the deep learning-based joint estimation model, and thus multiple joints corresponding to the user's posture in each image may be represented in a 2D coordinate system.

Here, the multiple joints may correspond to nine joints corresponding to the head, right shoulder, left shoulder, right hip, left hip, right knee, left knee, right foot, and left foot.

That is, according to the present disclosure, the positions of nine joints corresponding to the user posture in each image may be estimated merely by allowing the user to input the captured images to the joint estimation model, and then be represented in the 2D coordinate system.

Here, the multi-angle 2D pose may correspond to data represented by connecting the multiple joints to each other in the 2D coordinate system.

For example, as illustrated in FIG. 3 or 6, the nine joints estimated through the joint estimation model may be represented by points on a 2D plane, and respective points are connected to each other in conformity with the body structure of the user, and thus the 2D poses 330 and 630 may be created.

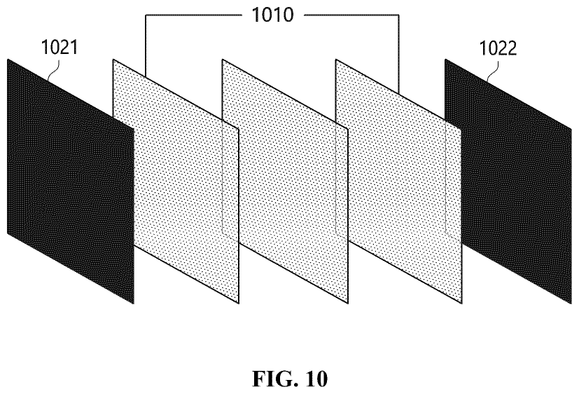

Here, because the images input to create the multi-angle 2D pose may be images captured by the user in his or her daily life, rather than images edited by an expert, unnecessary frames 1021 and 1022 in the start and end portions of images may be included, as illustrated in FIG. 10.

For example, unnecessary portions, such as portions captured to adjust a camera angle after pressing a capture start button of the camera, or portions moved to press the capture end button of the camera, may be included in the images.

In the present disclosure, in order to prevent computation from being performed on such unnecessary frames and then an unnecessary load from occurring in the system, valid frames 1010 may be extracted by deleting the unnecessary frames 1021 and 1022 from all frames corresponding to an image, and only the valid frames 1010 may be converted into array data.

In an example, the present disclosure may provide an image editing function so that the user personally selects only valid frames through a user interface provided to the user.

In another example, among the images, portions of images, in which the whole body of the user is not captured, may be determined to be unnecessary frames, and may be deleted.

In this case, a method for deleting unnecessary frames from images and extracting only valid frames may be implemented using various methods applicable to the present disclosure, and is not limited to a specific method.

Furthermore, the method for providing center-of-gravity information using an image according to an embodiment of the present disclosure may create a 3D pose by combining multi-angle 2D poses based on the posture information of the camera which captures images at step S120.

For example, 2D coordinates (X_{f}, Y_{f}) respectively corresponding to multiple joints may be obtained from the 2D pose for a front image f, included in the multi-angle 2D pose, and 2D coordinates (Xₛ, Yₛ) respectively corresponding to the multiple joints may be obtained from the 2D pose for a side image s included in the multi-angle 2D pose. Respective 2D coordinates obtained in this way may be combined with each other for respective joint positions depending on the posture of the camera, and thus 3D coordinates (X, Y, Z=X_{f}, Y_{f}, Xₛ) for respective joint positions may be obtained. In this case, the Z value of the 3D coordinates may correspond to the X value (Xₛ) of the 2D coordinates corresponding to the 2D pose for the side image s.

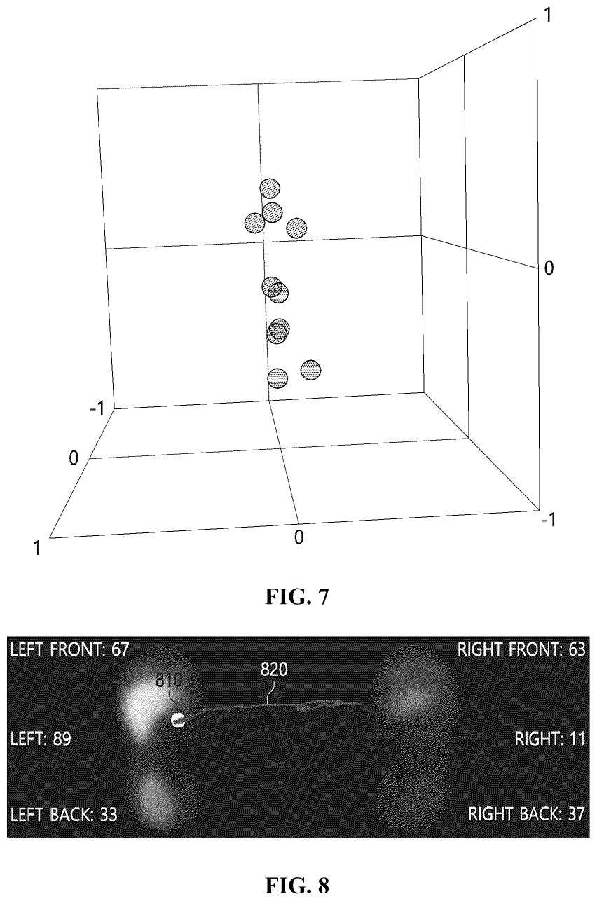

In this case, the obtained 3D coordinates may be scaled based on the head and tiptoes of the user, and thus the 3D coordinates may be represented to correspond to vector values in the 3D coordinate system, as illustrated in FIG. 7, with the result that a 3D pose corresponding to the user posture in the corresponding image may be created.

For example, the scaling of 3D coordinates may be performed in accordance with Equation 1.

$$µ = \{ max ( {Y}_{fLfoot} , {Y}_{fRfoot} ) + {Y}_{f\_head} \} / 2 \qquad [ Equation 1 ]$$

$$σ = \{ max ( {Y}_{fLfoot} , {Y}_{fRfoot} ) - {Y}_{f\_head} \}$$

$${ω}_{}^{*} = \frac{ω - µ}{σ}$$

Further, the method for providing center-of-gravity information using an image according to the embodiment of the present disclosure may generate a heatmap for visually showing a plantar pressure distribution based on the 3D pose, and may extract a midpoint on a weight balance based on the heatmap at step S130.

Here, the heatmap may be configured to show the plantar pressure distribution depending on the posture of the user so that the pressure distribution is visually identified, and may be generally provided in such a way that, when the user steps on a foothold provided with a pressure sensor, the center of gravity is measured based on a computation device connected to the pressure sensor.

However, in the present disclosure, although there is no pressure sensor, the heatmap may be generated by inputting the 3D pose created based on images to a deep learning-based center-of-gravity estimation model.

Here, the center-of-gravity estimation model may be a deep learning-based artificial neural network.

For example, the center-of-gravity estimation model according to an embodiment of the present disclosure may be a deep learning model-based artificial neural network designed by utilizing a TensorFlow framework.

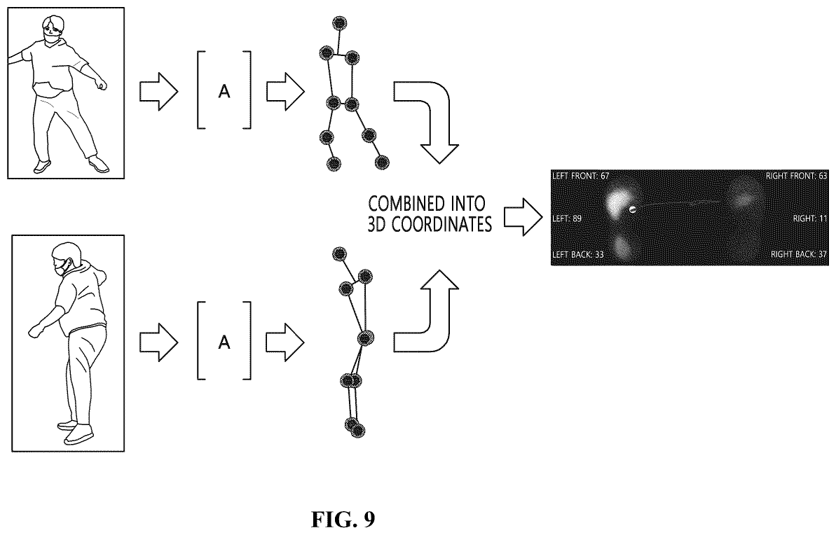

In this case, because both the front pose and the side pose of the user in the images may be known according to the previously created 3D pose, the front/rear and left/right weight balance of the user posture in the images may be estimated by inputting the 3D pose to the center-of-gravity estimation model. The front/rear and left/right weight balances estimated in this way are represented by pressure applied to both soles of the user, whereby the heatmap such as that shown in FIG. 8 may be provided.

In this case, the heatmap may be displayed such that, when the pressure applied to the soles is higher, the corresponding portion is visually emphasized.

For example, when the left/right weight balance indicated by numerical values in the heatmap illustrated in FIG. 8 is checked, LEFT is 89 and RIGHT is 11, and thus it can be seen that the weight balance is tilted to the left. Therefore, in order to clearly check at a glance that higher pressure is applied to the left foot than the right foot, the left foot may be more highlighted than the right foot on the heatmap.

FIG. 9 illustrates the above-described process to be seen at a glance, wherein respective 2D poses for a front image and a side image of a user in images may be created based on images captured from various angles, and a heatmap may be generated using a 3D pose created by combining the 2D poses with each other.

Furthermore, the method for providing center-of-gravity information using an image according to the embodiment of the present disclosure provides center-of-gravity information corresponding to the posture of the user using the weight balance calculated based on the midpoint on the weight balance at step S140.

Here, the weight balance may include a front/rear weight balance and a left/right weight balance.

Here, the midpoint on the weight balance may be extracted by normalizing output values from the heatmap.

For example, the midpoint on the weight balance (X^{p}, Y^{p}) may be output by applying a weighted Argsoftmax function, which is one of softmax functions, to the heatmap, as shown in the following Equation 2, and then normalizing the output values.

X^{p},Y^{p}=argsoftmax(3*heatmap) [Equation 2]

Here, correction values for the midpoint on the weight balance may be generated, the left/right weight balance of the left foot and the right foot of the user may be calculated based on the correction values for the midpoint on the weight balance, and front/fear weight balances corresponding to the left foot and the right foot, respectively, may be calculated by normalizing values corresponding to the left foot and the right foot, respectively, in the heatmap, extracting a midpoint on the weight balance of the left foot and a midpoint on the weight balance of the right foot, and correcting the extracted midpoints, respectively.

In an example, assuming that the correction values for the midpoint on the weight balance are (X^{p}, Y^{p}), left and right values, which correspond to the left/right weight balance, may be calculated using the following Equation 3.

right=100*(X^{p}−width*0.25)/(width*0.5)

left=100−right [Equation 3]

In another example, a midpoint on the weight balance of the left foot and a midpoint on the weight balance of the right foot may be respectively calculated by applying the weighted Argsoftmax function to each of the left foot and the right foot in the heatmap, and the front/rear weight balances corresponding to the left foot and the right foot, respectively, may be calculated based on values obtained by correcting the midpoint on the weight balance of the left foot and the midpoint on the weight balance of the right foot.

Assuming that the correction value for the midpoint on the weight balance of the left foot is Y^{L}^{_{_p}} and the correction value for the midpoint on the weight balance of the right foot is Y^{R}^{_{_p}}, R/L_front and R/L_rear, which are front/rear weight balances corresponding to the left foot and the right foot, may be calculated using the following Equation 4.

R/L_front=100*(Y^{R/L_P}−height*0.21)/(height*0.42)

R/L_rear=100−R/L_front [Equation 4]

Further, although not illustrated in FIG. 1, the method for providing center-of-gravity information using an image according to the embodiment of the present disclosure may output the center-of-gravity information to correspond to an image based on the user interface.

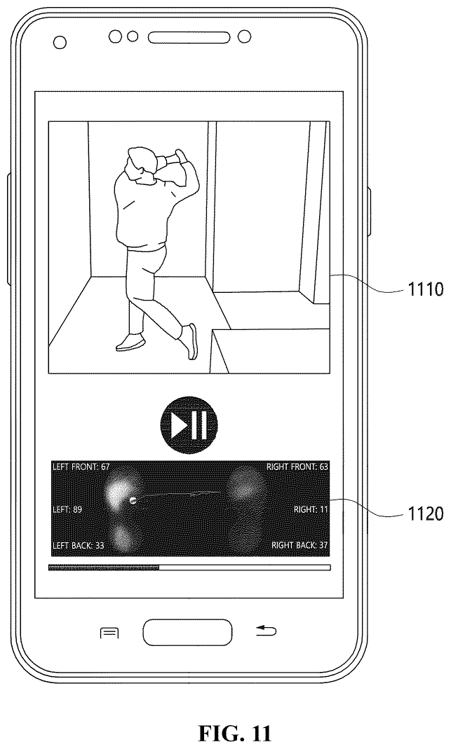

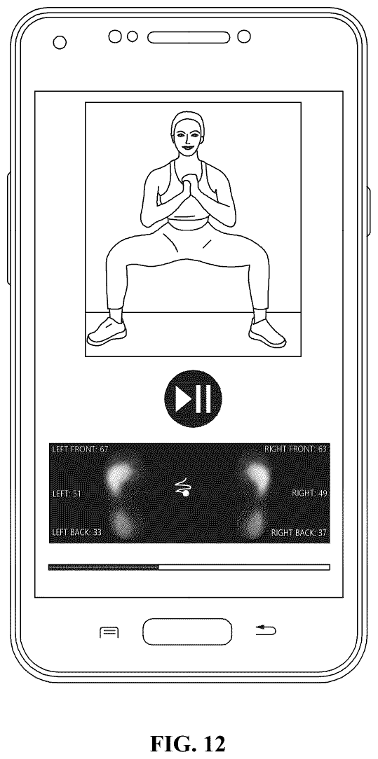

For example, referring to FIGS. 11 and 12, the user interface may be provided to the mobile terminal of the user, such as a smartphone. Here, the user interface may provide center-of-gravity information 1120 to match played images while playing images (video) 1110 used to output the center-of-gravity information.

That is, as illustrated in FIG. 11, center-of-gravity information related to a switching posture in golf may be provided, thus allowing the user to objectively understand the timing at which the center of gravity is to be shifted in golf swing and the degree of shift of the center of gravity. Further, a body part in a slightly incorrect posture may be analyzed through the extracted joint information, and the corresponding information may be provided to the user, thus enabling the information to be utilized for swing coaching.

Alternatively, as illustrated in FIG. 12, the corresponding information may be used as a basis for determining whether the motion of freehand exercise is exactly performed, or may be utilized for home workout coaching by analyzing the posture from joint information extracted when the user assumes home workout posture.

In this case, through the user interface, center-of-gravity information corresponding to at least one of the heatmap, the weight balance, the midpoint on the weight balance, and the shift line of the midpoint on the weight balance may be output.

For example, as illustrated in FIG. 8, a heatmap, a midpoint 810 on a weight balance represented on the heatmap, and a shift line 820 of the midpoint on the weight balance may be output, and front/rear weight balances of both feet, together with the left/right weight balance, may be digitized and output.

In this way, the center-of-gravity information provided according to the present disclosure may be serviced in the form of a PC or smartphone-based application without using a special output device.

Further, although not illustrated in FIG. 1, the method for providing center-of-gravity information using an image according to the embodiment of the present disclosure may store various types of information generated during the above-described process of providing the center-of-gravity information in a separate storage module.

By means of the method for providing center-of-gravity information, the center-of-gravity information related to the posture and balance of a user may be provided using only images captured by a camera in a usual place in which separate measurement equipment is not provided without restrictions in a place or a device.

Further, a center-of-gravity information service may be provided in the form of an application freely executable in an environment such as in a user's individual PC or a smartphone.

Furthermore, there can be provided a service that assists users who enjoy sports influenced by exercise posture or the shift of the center of gravity in correcting their postures or in more efficiently moving by providing center-of-gravity information to correspond to the motion of each user included in images.

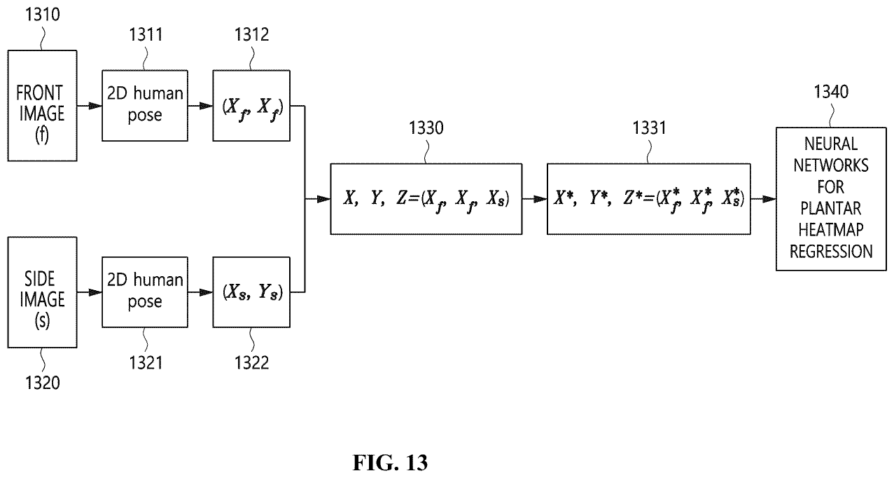

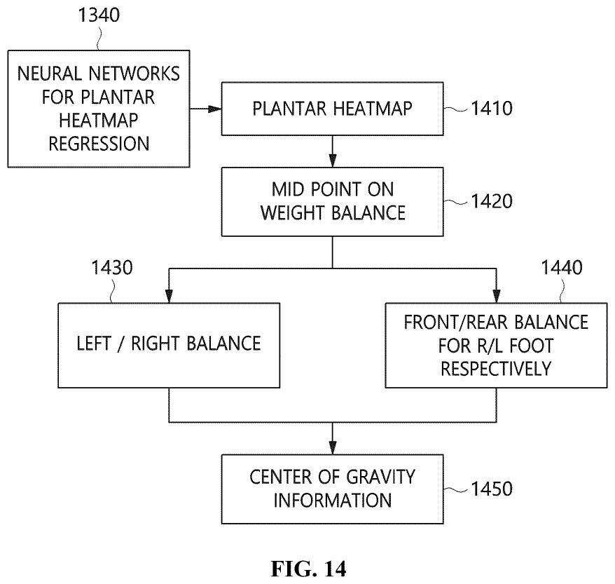

FIGS. 13 and 14 are diagrams illustrating in detail a process of providing center-of-gravity information according to an embodiment of the present disclosure.

First, referring to FIG. 13, the present disclosure may acquire a front image (f) 1310, obtained by capturing a user from the front, and a side image (s) 1320, obtained by capturing the user from the side, based on images obtained by capturing the user from various angles.

Thereafter, 2D poses 1311 and 1321 by which the positions of multiple joints are recognized may be created based on respective images.

In this case, each image may be converted into array data composed of 8-bit integers, and the array data may be input to a deep learning-based joint estimation model, and thus the 2D poses 1311 and 1321 in which multiple joints are represented in a 2D coordinate system may be created.

Thereafter, respective 3D coordinates 1330 of the multiple joints may be obtained by combining respective 2D coordinates 1312 and 1322 for the multiple joints, and a 3D pose may be created to correspond to 3D coordinates 1331 corrected by scaling the 3D coordinates 1330 based on the head and tiptoes.

Thereafter, a heatmap may be output by inputting the corrected 3D coordinates 1331 to a center-of-gravity estimation model 1340 corresponding to a neural network for generating a plantar heatmap.

Thereafter, referring to FIG. 14, a midpoint 1420 on a weight balance may be output by normalizing a heatmap 1410.

Thereafter, the left/right weight balance 1430 of the left foot and the right foot may be calculated based on a corrected value for the midpoint 1420 on the weight balance.

Furthermore, the center of gravity for the left foot and the center of gravity for the right foot may be extracted by normalizing values corresponding to the left foot and the right foot, respectively, in the heatmap, and front/rear weight balances 1440 respectively corresponding to the left foot and the right foot may be calculated by correcting the extracted centers of gravity.

Thereafter, center-of-gravity information 1450 to be provided to the user may be generated based on the generated heatmap, the calculated midpoint on the weight balance, and the weight balances.

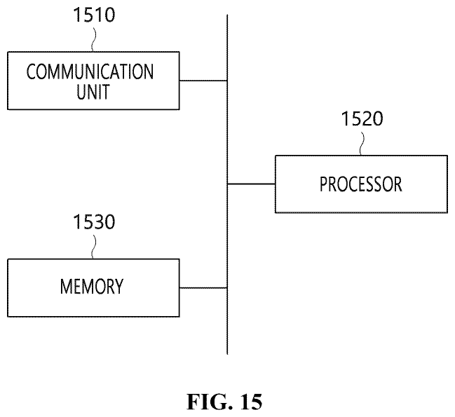

FIG. 15 is a block diagram illustrating an apparatus for providing center-of-gravity information using an image according to an embodiment of the present disclosure.

Referring to FIG. 15, the apparatus for providing center-of-gravity information using an image according to the embodiment of the present disclosure includes a communication unit 1510, a processor 1520, and memory 1530.

The communication unit 1510 functions to transmit/receive information required for providing center-of-gravity information over a communication network such as a network.

Here, the network is a concept including both networks that have been conventionally used and networks that may be developed in the future. For example, such a network may be any one of a wired/wireless LAN for providing communication between various types of information devices in a limited area, a mobile communication network for providing communication between individual moving objects and between a moving object and an external system outside the moving object, a satellite communication network for providing communication between individual earth stations using satellites, and a wired/wireless communication network, or a combination of two or more thereof. Meanwhile, transfer mode standards for the network are not limited to existing transfer mode standards, but may include all transfer mode standards to be developed in the future.

The processor 1520 may generate multi-angle 2D poses corresponding to the posture of a user based on images acquired by capturing the user from various angles.

That is, the present disclosure may create a 2D pose used to generate center-of-gravity information by utilizing only images captured through a camera even in a usual place in which separate center-of-gravity measurement equipment is not provided.

For example, when the user desires to know whether his or her posture or balance is correct while doing workouts at home, the user may capture images of his or her home workout postures using a camera provided in the smartphone of the user, and may input the captured home workout images through a user interface provided to the smartphone according to an embodiment of the present disclosure.

Here, the images captured from various angles may include a front image obtained by capturing the user from the front and a side image obtained by capturing the user from the side.

Therefore, a multi-angle 2D pose may include a 2D pose created to correspond to the front posture of the user extracted from the front image and a 2D pose created to correspond to the side posture of the user extracted from the side image.

Here, in the present disclosure, the left/right weight balance of the user may be estimated using the 2D pose created to correspond to the front posture of the user.

For example, as illustrated in FIG. 2, when the user is captured using one camera 200 located in front of the user, an image 310 including only the front posture of the user may be acquired, as illustrated in FIG. 3. Here, through a process of converting the image 310 into array data 320, which will be described later, a 2D pose 330 corresponding to the front posture of the user may be created.

When the 2D pose 330 created in this way is used, the left/right weight balance 400 of the front posture of the user may be estimated, as illustrated in FIG. 4. A process of estimating the left/right weight balance using the 2D pose corresponding to the front posture of the user will be described in detail later.

Here, the left/right weight balance 400 illustrated in FIG. 4 may be a value estimated based on the feet of the user captured in the image 310, illustrated in FIG. 3, and the balance of a right foot and the balance of a left foot may be estimated to be values in inverse proportion to each other.

For example, when the body of the user is analyzed as tilting to the right based on the 2D pose, the balance value of the left foot may be decreased instead of the balance value of the right foot being increased. On the other hand, when the body of the user is analyzed as tilting to the left based on the 2D pose, the balance value of the right foot may be decreased instead of the balance value of the left foot being increased.

Further, in the present disclosure, the front/rear weight balance of the user may also be estimated using the 2D pose created to correspond to the side posture of the user.

For example, as illustrated in FIG. 5, when the user is simultaneously captured using a camera 510 located in front of the user and a camera 520 located on the side of the user, an image 610 corresponding to the side posture of the user at the same time point, as shown in FIG. 6, may be acquired together with the image 310 corresponding to the front posture of the user, shown in FIG. 3. In this case, when the image 610 corresponding to the side posture of the user undergoes the process of conversion into array data 620, which will be described later, a 2D pose 630 corresponding to the side posture of the user may be created.

That is, a multi-angle 2D pose in the present disclosure may be a concept including both the 2D pose 330 corresponding to the front posture, illustrated in FIG. 3, and the 2D pose 630 corresponding to the side posture, illustrated in FIG. 6.

Here, in order to create the multi-angle 2D pose, the positions of respective joints of the user need to be estimated by inputting images captured from various angles to a deep learning-based joint estimation model, wherein, for this operation, the images captured from various angles may be first converted into array data to be input to the deep learning-based joint estimation model.

Here, the joint estimation model may correspond to a deep learning-based model.

For example, the joint estimation model according to an embodiment of the present disclosure may correspond to a deep learning model designed by utilizing a TensorFlow framework.

Here, the array data may be data composed of 8-bit integers.

Here, the images converted into the array data may be input to the deep learning-based joint estimation model, and thus multiple joints corresponding to the user's posture in each image may be represented in a 2D coordinate system.

Here, the multiple joints may correspond to nine joints corresponding to the head, right shoulder, left shoulder, right hip, left hip, right knee, left knee, right foot, and left foot.

That is, according to the present disclosure, the positions of nine joints corresponding to the user posture in each image may be estimated merely by allowing the user to input the captured images to the joint estimation model, and then be represented in the 2D coordinate system.

Here, the multi-angle 2D pose may correspond to data represented by connecting the multiple joints to each other in the 2D coordinate system.

For example, as illustrated in FIG. 3 or 6, the nine joints estimated through the joint estimation model may be represented by points on a 2D plane, and respective points are connected to each other in conformity with the body structure of the user, and thus the 2D poses 330 and 630 may be created.

Here, because the images input to create the multi-angle 2D pose may be images captured by the user in his or her daily life, rather than images edited by an expert, unnecessary frames 1021 and 1022 in the start and end portions of images may be included, as illustrated in FIG. 10.

For example, unnecessary portions, such as portions captured to adjust a camera angle after pressing a capture start button of the camera, or portions moved to press the capture end button of the camera, may be included in the images.

In the present disclosure, in order to prevent computation from being performed on such unnecessary frames and then an unnecessary load from occurring in the system, valid frames 1010 may be extracted by deleting the unnecessary frames 1021 and 1022 from all frames corresponding to an image, and only the valid frames 1010 may be converted into array data.

In an example, the present disclosure may provide an image editing function so that the user personally selects only valid frames through a user interface provided to the user.

In another example, among the images, portions of images, in which the whole body of the user is not captured, may be determined to be unnecessary frames, and may be deleted.

In this case, a method for deleting unnecessary frames from images and extracting only valid frames may be implemented using various methods applicable to the present disclosure, and is not limited to a specific method.

Further, the processor 1520 may create a 3D pose by combining the multi-angle 2D poses based on the posture information of the camera which captures the images.

For example, 2D coordinates (X_{f}, Y_{f}) respectively corresponding to multiple joints may be obtained from the 2D pose for a front image f, included in the multi-angle 2D pose, and 2D coordinates (Xₛ, Yₛ) respectively corresponding to the multiple joints may be obtained from the 2D pose for a side image s included in the multi-angle 2D pose. Respective 2D coordinates obtained in this way may be combined with each other for respective joint positions depending on the posture of the camera, and thus 3D coordinates (X, Y, Z=X_{f}, Y_{f}, Xₛ) for respective joint positions may be obtained. In this case, the Z value of the 3D coordinates may correspond to the X value (Xₛ) of the 2D coordinates corresponding to the 2D pose for the side image s.

In this case, the obtained 3D coordinates may be scaled based on the head and tiptoes of the user, and thus the 3D coordinates may be represented to correspond to vector values in the 3D coordinate system, as illustrated in FIG. 7, with the result that a 3D pose corresponding to the user posture in the corresponding image may be created.

For example, the scaling of 3D coordinates may be performed in accordance with Equation 1.

$$µ = \{ max ( {Y}_{fLfoot} , {Y}_{fRfoot} ) + {Y}_{f\_head} \} / 2 \qquad [ Equation 1 ]$$

$$σ = \{ max ( {Y}_{fLfoot} , {Y}_{fRfoot} ) - {Y}_{f\_head} \}$$

$${ω}_{}^{*} = \frac{ω - µ}{σ}$$

Further, the processor 1520 may generate a heatmap for visually showing a plantar pressure distribution based on the 3D pose, and may extract a midpoint on weight balance based on the heatmap.

Here, the heatmap may be configured to show the plantar pressure distribution depending on the posture of the user so that the pressure distribution is visually identified, and may be generally provided in such a way that, when the user steps on a foothold provided with a pressure sensor, the center of gravity is measured based on a computation device connected to the pressure sensor.

However, in the present disclosure, although there is no pressure sensor, the heatmap may be generated by inputting the 3D pose created based on images to a deep learning-based center-of-gravity estimation model.

Here, the center-of-gravity estimation model may be a deep learning-based artificial neural network.

For example, the center-of-gravity estimation model according to an embodiment of the present disclosure may be a deep learning model-based artificial neural network designed by utilizing a TensorFlow framework.

In this case, because both the front pose and the side pose of the user in the images may be known according to the previously created 3D pose, the front/rear and left/right weight balance of the user posture in the images may be estimated by inputting the 3D pose to the center-of-gravity estimation model. The front/rear and left/right weight balances estimated in this way are represented by pressure applied to both soles of the user, whereby the heatmap such as that shown in FIG. 8 may be provided.

In this case, the heatmap may be displayed such that, when the pressure applied to the soles is higher, the corresponding portion is visually emphasized.

For example, when the left/right weight balance indicated by numerical values in the heatmap illustrated in FIG. 8 is checked, LEFT is 89 and RIGHT is 11, and thus it can be seen that the weight balance is tilted to the left. Therefore, in order to clearly check at a glance that higher pressure is applied to the left foot than the right foot, the left foot may be more highlighted than the right foot on the heatmap.

FIG. 9 illustrates the above-described process to be seen at a glance, wherein respective 2D poses for a front image and a side image of a user in images may be created based on images captured from various angles, and a heatmap may be generated using a 3D pose created by combining the 2D poses with each other.

Furthermore, the processor 1520 provides center-of-gravity information corresponding to the posture of the user using the weight balance calculated based on the midpoint on weight balance.

Here, the weight balance may include a front/rear weight balance and a left/right weight balance.

Here, the midpoint on the weight balance may be extracted by normalizing output values from the heatmap.

For example, the midpoint on the weight balance (X′, Y′) may be output by applying a weighted Argsoftmax function, which is one of softmax functions, to the heatmap, as shown in the following Equation 2, and then performing normalization.

X^{p},Y^{p}=argsoftmax(3*heatmap) [Equation 2]

Here, correction values for the midpoint on the weight balance may be generated, the left/right weight balance corresponding to the left foot and the right foot of the user may be calculated based on the correction values for the midpoint on the weight balance, and front/fear weight balances corresponding to the left foot and the right foot, respectively, may be calculated by normalizing values corresponding to the left foot and the right foot, respectively, in the heatmap, extracting a midpoint on the weight balance of the left foot and a midpoint on the weight balance of the right foot, and correcting the extracted midpoints, respectively.

In an example, assuming that the correction values for the midpoint on the weight balance are (X^{p}, Y^{p}), left and right values, which correspond to the left/right weight balance, may be calculated using the following Equation 3.

right=100*(X^{p}−width*0.25)/(width*0.5)

left=100−right [Equation 3]

In another example, a midpoint on the weight balance of the left foot and a midpoint on the weight balance of the right foot may be respectively calculated by applying the weighted Argsoftmax function to each of the left foot and the right foot in the heatmap, and the front/rear weight balances corresponding to the left foot and the right foot, respectively, may be calculated based on values obtained by correcting the midpoint on the weight balance of the left foot and the midpoint on the weight balance of the right foot.

Assuming that the correction value for the midpoint on the weight balance of the left foot is Y^{L}^{_{_p}} and the correction value for the midpoint on the weight balance of the right foot is Y^{R}^{_{_p}}, R/L_front and R/L_rear, which are front/rear weight balances corresponding to the left foot and the right foot, may be calculated using the following Equation 4.

R/L_front=100*(Y^{R/L_P}−height*0.21)/(height*0.42)

R/L_rear=100−R/L_front [Equation 4]

Further, the processor 1520 outputs the center-of-gravity information to correspond to images based on a user interface.

For example, referring to FIGS. 11 and 12, the user interface may be provided to the mobile terminal of the user, such as a smartphone. Here, the user interface may provide center-of-gravity information 1120 to match played images while playing images 1110 used to output the center-of-gravity information.

That is, as illustrated in FIG. 11, center-of-gravity information related to a switching posture in golf may be provided, thus allowing the user to objectively understand the timing at which the center of gravity is to be shifted in golf swing and the degree of shift of the center of gravity. Further, a body part in a slightly incorrect posture may be analyzed through the extracted joint information, and the corresponding information may be provided to the user, thus enabling the information to be utilized for swing coaching.

Alternatively, as illustrated in FIG. 12, the corresponding information may be used as a basis for determining whether the motion of freehand exercise is exactly performed, or may be utilized for home workout coaching by analyzing the posture from joint information extracted when the user assumes home workout posture.

In this case, through the user interface, center-of-gravity information corresponding to at least one of the heatmap, the weight balance, the midpoint on the weight balance, and the shift line of the midpoint on the weight balance may be output.

For example, as illustrated in FIG. 8, a heatmap, a midpoint 810 on a weight balance represented on the heatmap, and a shift line 820 of the midpoint on the weight balance may be output, and front/rear weight balances of both feet, together with the left/right weight balance, may be digitized and output.

The memory 1530 stores at least one of the images, the heatmap, or the center-of-gravity information.

Further, the memory 1530 may store various types of information generated during a process of providing the center-of-gravity information.

By means of the apparatus for providing center-of-gravity information, center-of-gravity information related to the posture and balance of a user may be provided using only images captured by a camera in a usual place in which separate measurement equipment is not provided without restrictions in a place or a device.

Further, there can be provided a center-of-gravity information service in the form of an application freely executable in an environment such as in a user's individual PC or a smartphone.

Furthermore, there can be provided a service that assists users who enjoy sports influenced by exercise posture or the shift of the center of gravity in correcting their postures or in more efficiently moving by providing center-of-gravity information to correspond to the motion of each user included in images.

As described above, in the method for providing center-of-gravity information using an image and the apparatus for the method according to the present disclosure, the configurations and schemes in the above-described embodiments are not limitedly applied, and some or all of the above embodiments can be selectively combined and configured so that various modifications are possible.

What is claimed is:

1. A method for providing center-of-gravity information, comprising:
creating multi-angle two-dimensional (2D) poses corresponding to posture of a user based on images obtained by capturing the user from various angles;
creating a three-dimensional (3D) pose by combining the multi-angle 2D poses with each other;
generating a heatmap for visually showing a plantar pressure distribution based on the 3D pose, and extracting a midpoint on a weight balance based on the heatmap; and
providing center-of-gravity information corresponding to the posture of the user using a weight balance calculated based on the midpoint on the weight balance.

2. The method of claim 1, wherein the weight balance comprises a front/rear weight balance and a left/right weight balance.

3. The method of claim 2, further comprising:
outputting the center-of-gravity information to correspond to the images based on a user interface.

4. The method of claim 3, wherein the outputting comprises:
outputting at least one of the heatmap, the weight balance, the midpoint on the weight balance, and a shift line of the midpoint on the weight balance.

5. The method of claim 1, wherein generating the multi-angle 2D poses comprises:
converting each image into array data composed of 8-bit integers; and
representing multiple joints corresponding to the posture of the user in a 2D coordinate system by inputting the array data into a deep learning-based joint estimation model.

6. The method of claim 5, wherein the multi-angle 2D poses correspond to data represented by connecting the multiple joints to each other in the 2D coordinate system.

7. The method of claim 1, wherein extracting the midpoint on the weight balance comprises:
generating the heatmap by inputting the 3D pose to a deep learning-based center-of-gravity estimation model.

8. The method of claim 7, wherein extracting the midpoint on the weight balance comprises:
extracting the midpoint on the weight balance by normalizing output values from the heatmap.

9. The method of claim 8, wherein providing the center-of-gravity information comprises:
generating a correction value for the midpoint on the weight balance;
calculating a left/right weight balance of a left foot and a right foot of the user based on the correction value for the midpoint on the weight balance; and
calculating front/fear weight balances corresponding to the left foot and the right foot by normalizing values corresponding to the left foot and the right foot, respectively, in the heatmap to extract a midpoint on a weight balance of the left foot and a midpoint on a weight balance of the right foot and by correcting respective extracted midpoints.

10. The method of claim 1, wherein the images captured from various angles include a front image obtained by capturing the user from front and a side image obtained by capturing the user from side.

11. The method of claim 5, wherein converting into the array data comprises:
extracting valid frames by deleting unnecessary frames from all frames corresponding to the images, and converting the valid frames into the array data.

12. The method of claim 1, wherein the heatmap is displayed to be visually highlighted as pressure applied to soles is higher.

13. An apparatus for center-of-gravity information, comprising:
a processor configured to create multi-angle two-dimensional (2D) poses corresponding to posture of a user based on images obtained by capturing the user from various angles, create a three-dimensional (3D) pose by combining the multi-angle 2D poses with each other, generate a heatmap for visually showing a plantar pressure distribution based on the 3D pose, extract a midpoint on a weight balance based on the heatmap, and provide center-of-gravity information corresponding to the posture of the user using a weight balance calculated based on the midpoint on the weight balance; and
a memory configured to store at least one of the images, the heatmap, and the center-of-gravity information.

14. The apparatus of claim 13, wherein the weight balance comprises a front/rear weight balance and a left/right weight balance.

15. The apparatus of claim 14, wherein the processor is configured to output the center-of-gravity information to correspond to the images based on a user interface.

16. The apparatus of claim 15, wherein the processor is configured to output at least one of the heatmap, the weight balance, the midpoint on the weight balance, and a shift line of the midpoint on the weight balance.

17. The apparatus of claim 13, wherein the processor is configured to convert each image into array data composed of 8-bit integers and represent multiple joints corresponding to the posture of the user in a 2D coordinate system by inputting the array data into a deep learning-based joint estimation model.

18. The apparatus of claim 17, wherein the multi-angle 2D poses correspond to data represented by connecting the multiple joints to each other in the 2D coordinate system.

19. The apparatus of claim 13, wherein the processor is configured to generate the heatmap by inputting the 3D pose to a deep learning-based center-of-gravity estimation model.

20. The apparatus of claim 19, wherein the processor is configured to extract the midpoint on the weight balance by normalizing output values from the heatmap.

21. The apparatus of claim 20, wherein the processor is configured to generate a correction value for the midpoint on the weight balance, calculate a left/right weight balance of a left foot and a right foot of the user based on the correction value for the midpoint on the weight balance, and calculate front/fear weight balances corresponding to the left foot and the right foot by normalizing values corresponding to the left foot and the right foot, respectively, in the heatmap to extract a midpoint on a weight balance of the left foot and a midpoint on a weight balance of the right foot and by correcting respective extracted midpoints.

22. The apparatus of claim 13, wherein the images captured from various angles include a front image obtained by capturing the user from front and a side image obtained by capturing the user from side.

23. The apparatus of claim 17, wherein the processor is configured to extract valid frames by deleting unnecessary frames from all frames corresponding to the images, and convert the valid frames into the array data.

24. The apparatus of claim 13, wherein the heatmap is displayed to be visually highlighted as pressure applied to soles is higher.
